(19) United States
(12) Patent Application Publication
(10) Pub. No.: US 2021/0087639 A1
(43) Pub. Date: Mar. 25, 2021

(54) Method of Treatment Utilizing a Gene Expression Signature Predicting the Response of HER2-Directed Therapies

(71) Applicant: Rush University Medical Center, Chicago, IL (US)

(72) Inventors: Abdelrahmman Mohammad ABUKHDEIR, Bolingbrook, IL (US); Melody COBLEIGH, Riverside, IL (US); Matthew NAJOR, Chicago, IL (US)

(73) Assignee: Rush University Medical Center, Chicago, IL (US)

(21) Appl. No.: 17/040,314

(22) PCT Filed: Mar. 22, 2019
(86) PCT No.: PCT/US19/23562

§ 371 Date: Sep. 22, 2020

Related U.S. Application Data
(60) Provisional application No. 62/648,055, filed on Mar. 26, 2018.

(51) Int. Cl. C12Q 1/6886; C07K 16/32

(52) U.S. Cl. CPC C12Q 1/6886; C07K 16/32; C12Q 2600/106; C12Q 2600/156; C12Q 2600/154; C12Q 2600/158

(57) ABSTRACT

The present invention generally relates to methods for predicting the response of HER2-directed therapies in cancer and to methods of treating the cancer based on the prediction. In one embodiment the cancer is breast cancer. In another embodiment, the method predicts the response to treatment with trastuzumab.

### RELATED APPLICATIONS

The present patent application claims the benefit of the filing date of U.S. Provisional Patent Application No. 62/648,055, filed Mar. 26, 2018, the contents of which is hereby incorporated by reference.

### TECHNICAL FIELD

The present invention generally relates to methods for treating a cancer. In one embodiment, the method includes predicting the response of HER2-directed therapies in the treatment of the cancer and administering a treatment based on the predicted response. In one embodiment the cancer is breast cancer. In another embodiment, the method predicts the response to treatment with trastuzumab.

### BACKGROUND

It is estimated that by the end of 2016, approximately 226,870 women will be newly diagnosed with breast cancer in the US alone, and 39,510 women will die from their disease. Of all breast cancers, approximately 15-20% over-express the avian erythroblastosis oncogene B 2 gene (ERBB2; also called HER2/neu), which is a membrane-bound protein with no known ligand (1). HER2+ breast cancers are among the most aggressive. Historically, this type of breast cancer has carried a very poor prognosis (1, 2). However, humanized, monoclonal antibodies directed against the ERBB2 protein improved the prognosis for women with this type of breast cancer (3).

The first commercially available antibody directed against the ERBB2 protein was trastuzumab (HERCEPTIN™). Trastuzumab entered clinical trials as a first-line monotherapy and produced a response rate of patients with HER2+ cancers from 14% to 15-50%. The response rate varied depending on the prior treatment (4-6). The promising results from these studies in the metastatic setting, led to the design of several clinical trials in the adjuvant setting using different combinations of trastuzumab and chemotherapeutics (7-9). Trastuzumab is now administered with pertuzumab in conjunction with chemotherapy in the adjuvant setting as the standard of care for most women diagnosed with HER2+ breast cancer (10).

Unfortunately, in the metastatic setting, the majority of tumors have either de novo or acquired resistance. Therefore, although HER2-directed treatment increases survival, the problem of resistance has generated an urgent need to determine the mechanisms of resistance. Several mechanisms of trastuzumab resistance have been proposed; however, there are several examples of discordance between the pre-clinical and clinical studies (11-13).

### SUMMARY

One aspect of the present invention provides a method for treating a cancer in a subject. In one embodiment, the method includes determining a gene signature based on expression, copy number, or promoter methylation levels of at least two genes in a sample taken from the subject. The genes are selected from the group consisting of ECM1, KAT2B, KMO, PBX3, STAT6 and ZBTB37. A treatment strategy is selected and administered to the subject based on the gene signature.

In other embodiments, the gene expression signature is based on expression levels of at least three, four, or five or all of the genes selected from the group consisting of ECM1, KAT2B, KMO, PBX3, STAT6 and ZBTB37. The treatment strategy may include a less harsh more targeted treatment strategy, such as single-agent trastuzumab therapy, when the gene expression signature is indicative of at least two, three, four, five, or all of: normal or decreased expression levels of ECM1, KAT2B, KMO, PBX3 and ZBTB37 and normal or increased expression level of STAT6.

In another embodiment, the treatment strategy includes administering a harsh non-targeted treatment strategy when the gene expression signature is indicative of at least two, three, four, five or all of: elevated expression levels of ECM1, KAT2B, KMO, PBX3 and ZBTB37 and decreased expression level of STAT6.

In certain embodiments, the harsh treatment strategy may include administering chemotherapy alone or, alternatively, trastuzumab in combination with pertuzumab and chemotherapy. The cancer may be breast cancer, for example, HER2+ breast cancer.

### BRIEF DESCRIPTION OF THE DRAWINGS

FIG. 1 illustrates immunoblotting of BT474 parent (P) and child (C) probed for HER2. MCF-10A (10A) cells were used as a negative control. β-Tubulin (TUBB) was used as a protein loading control.

FIG. 2 is a plot showing the number of genetic alterations from the six gene signature plotted against mean overall survival (months).

FIG. 3 is a chart showing a Kaplan Meier plot of those with ≤1 alteration (solid line) and ≥2 alterations (dashed line) in the HER2+ population (N=120) of TOGA Cell Study, p<0.05.

FIG. 4 is a chart showing a Kaplan Meier plot of those with ≤1 alteration (solid line) and ≥2 alterations (dashed line) in the HER2+ population (N=49) of TOGA Nature Study, p<0.05.

FIG. 5 is a chart showing a Kaplan Meier of the 6-gene signature validated in the HER2+ population (N=150) in KM Plotter, p<0.05, (solid line) is survival of patients with low expression and (dashed line) is survival of patients with high expression.

FIG. 6 is a bar plot of the expression of the six candidate genes as determined by qPCR validation. All values are relative to the control, BT474 parent. Values >1 and <1 indicate upregulation or downregulation of gene expression in child, respectively. Expression is calculated from the ratio of efficiencies raised to the power of ΔCq for gene of interest over reference. Expression values are means of three independent experiments.

FIG. 7 are graphs of individual Kaplan Meier plots for four candidate genes.

FIG. 8 are graphs of individual Kaplan Meier plots for an additional four candidate genes.

FIG. 9 are graphs of Kaplan Meier plots for genes validated in Cell 2015 Study (27), HER2+ population N=120, p<0.05, (dashed line) is survival of patients with gene alterations and (solid line) is survival of patients without gene alterations.

FIG. 10 are graphs of Kaplan Meier plots for genes validated in Cell 2015 Study (27), HER2+ population N=120, p<0.05, (dashed line) is survival of patients with gene alterations and (solid line) is survival of patients without gene alterations.

FIG. 11 is a Kaplan Meier plot of overall survival (OS) of 6-signature as validated in HER2+ population (N=120), p<0.05, of Cell 2015 study. (dashed line) is survival of patients with gene alterations and (solid line) is survival of patients without gene alterations.

### DETAILED DESCRIPTION

### Definitions

For the purpose of promoting an understanding of the principles of the invention, reference will now be made to embodiments, some of which are illustrated in the drawings, and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. In the discussions that follow, a number of potential features or selections of assay methods, methods of analysis, or other aspects, are disclosed. It is to be understood that each such disclosed feature or features can be combined with the generalized features discussed, to form a disclosed embodiment of the present invention.

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention.

The uses of the terms “a” and “an” and “the” and similar references in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”, “for example”) provided herein, is intended merely to better illuminate the invention and does not pose a limitation on the scope of the invention unless otherwise claimed. No language in the specification should be construed as indicating any non-claimed element as essential to the practice of the invention.

The term “therapeutic effect” as used herein means an effect which induces, ameliorates or otherwise causes an improvement in the pathological symptoms, disease progression or physiological conditions associated with a disorder, for example cancer, of a human or veterinary subject. The term “therapeutically effective amount” as used with respect to a drug means an amount of the drug which imparts a therapeutic effect to the human or veterinary subject.

Methods of Treating a Cancer

One aspect of the present invention provides a method for treating a cancer in a human or veterinary subject. The cancer may be any cancer with elevated expression or copy number of HER2, including HER2+ breast cancer. Other examples of cancers that may be treated using this method include bladder cancer, carcinoid cancer, colon cancer, ductal carcinoma in situ (DCIS), endometrium cancer, head/neck cancer, kidney cancer, lung cancer, melanoma, ovarian cancer, phylloides cancer, prostate cancer, stomach cancer, testicular cancer and thyroid cancer. In one embodiment, the method includes testing a sample from the subject and basing a treatment strategy on the outcome of the test. For example, the test may be used to distinguish those subjects who are likely to respond to a first treatment (less harsh) strategy from those who require a harsh treatment strategy. The harsh treatment strategy may be one that includes the administration of highly cytotoxic drugs, whereas the less harsh treatment may avoid the administration of such drugs. In one embodiment, the method includes the steps of predicting the response to treatment with a therapeutic, such as trastuzumab, before the treatment is administered and deciding on the treatment strategy based on the prediction. In one embodiment, the method allows for the determination of those cancers that are more likely respond to single-agent trastuzumab therapy or to the use of multiple inhibitors of HER2 simultaneously.

The advent of trastuzumab, sometimes in combination with other inhibitors of HER2, has dramatically improved the outcomes of subjects with cancers, such as HER2 positive breast cancers. However, not all subjects will respond to trastuzumab and most subjects will eventually recur with trastuzumab-resistant metastatic breast cancer. Approximately 20% of subjects will experience an exquisitely sensitive response to single-agent trastuzumab, but, before the present invention, there was no way to know who will experience such a response. Therefore, all subjects are indiscriminately given chemotherapy, including the administration of highly cytotoxic drugs, concurrently with trastuzumab. The present method allows for the identification of those subjects who will respond to single-agent trastuzumab therapy, inhibitors of HER2 or other, less harsh, treatment strategies. Subjects may be separated into two treatment groups. A first group of subjects may be treated without the administration of highly cytotoxic drugs, for example, using single agent trastuzumab therapy. A second group of subjects may be treated using a harsh treatment strategy, including the administering of chemotherapy using highly cytotoxic drugs.

In one embodiment, the present method includes determining a gene expression signature from a sample obtained from the subject. For example, the sample may be a blood sample, a tissue sample, or a sample taken from a tumor. The signature is based on expression levels, copy number, or promoter methylation levels of at least two genes selected from the group including of ECM1, KAT2B, KMO, PBX3, STATE and ZBTB37. For example, expression levels, copy number, or promoter methylation levels of two, three, four, five or six of these genes are determined.

A treatment is then administered to the subject based on the gene expression signature. In other embodiments, the treatment strategy is based on expression levels, copy number, or promoter methylation levels of at least three, four, five or all of these genes.

The present method allows for the separation of subjects into a first and a second category. The first category includes those subjects likely to be successfully treated by a less harsh treatment strategy. Such treatment may include the administration of trastuzumab in the absence of another anti-cancer drug (single agent trastuzumab therapy). Other example of such treatments include treatment with multiple HER2-targeted agents simultaneously, such as ado trastuzumab, emtansine, bevacizumab, everolimus, palbociclib, trastuzumab, pertuzumab, lapatinib, or neratinib. The HER2-targeted agents may in some cases be supplemented with estrogen-receptor-targeted agents such as tamoxifen, zoladex, oorporectomy, anastrozole, letrozole, faslodex, palbociclib, exemestane+everolimus10 mg daily, high dose estradiol, megace, or halotestin.

The second category includes those subjects whose cancer is likely resistant to such treatments. Subjects in the second category may be treated with a harsh treatment strategy. Examples of such a treatment strategy include, but are not limited to, chemotherapy using one or more drugs such as doxorubicin, cyclophosphamide, doxil, eribulin mesylate, ixabepilone, vinorelbine, gemcitabine, cis-platinum, carboplatin, nab-paclitaxel, paclitaxel, docetaxel, capecitabine, metronomic methotrexate, cyclophosphamide, oral VP-16 and bevacizumab.

In one embodiment, elevated expression or copy number, or reduced promotor methylation, of the genes ECM1, KAT2B, KMO, PBX3 and ZBTB37 and/or decreased expression or copy number, or increased promotor methylation, of the STAT6 gene predict the overall survival of subjects whose cancers express amplified and/or elevated expression levels of the ERBB2. Such subjects are considered less likely to respond when treated without the administration of highly cytotoxic drugs (i.e. using a less harsh treatment strategy) and are treated using a harsh treatment strategy.

In other embodiments, the method includes administering a harsh treatment strategy when two, three, four, five or all of the following conditions are found to be present in a sample obtained from the subject: increased expression or copy number, or decreased promotor methylation, of ECM1, KAT2B, KMO, PBX3, ZBTB37; decreased expression or copy number, or increased promotor methylation of STAT6. The harsh treatment strategy may be any of those harsh treatments disclosed above, for example, administration of a therapeutically effective amount of a combination of trastuzumab and a cytotoxic drug chemotherapy, or in combination with pertuzumab and/or chemotherapy. In other embodiments, chemotherapy with cytotoxic drug(s) is used.

### EXAMPLES

### Example 1—Cell Culture

Cellular clones of the BT474 cell line were a kind gift from Dr. Susan Kane at City of Hope National Medical Center (Duarte, Calif.). Clones were maintained in RPMI Media (Life Technologies, Grand Island, N.Y.) supplemented with 5% Fetal Bovine Serum (FBS) (Sigma, Saint Louis, Mo.), 1 mM Sodium Pyruvate (Life Technologies, Grand Island, N.Y.), 5 g/L Glucose (Sigma, Saint Louis, Mo.) and 1% Penicillin/Streptomycin (Life Technologies, Grand Island, N.Y.). Drug resistant clones were grown in the same conditions, except the medium was supplemented with 1 μM trastuzumab (Roche, San Francisco, Calif.) to maintain drug resistance.

### Example 2—RNA Sequencing

Total RNA from cellular clones was extracted as previously described (16). RNA integrity was validated using an RNA analysis screentape, implemented on a TapeStation2200 (Agilent). Ribosomal RNA was depleted using the Low Input RiboMinus™ Eukaryote System v2 (Life Technologies). Depleted RNA was prepared for sequencing using the directional PrepX RNASeq library preparation kit, implemented on the Apollo324 NGS. The final library fragment sizes ranged from 80-900 bp, with an average of 400 bp. The libraries were pooled in equimolar concentration and the pool was sequenced on an lllumina HiSeq2500 sequencer using a TruSeq Rapid SBS sequencing kit version 1. Paired-end reads were 165 nucleotides in length. Demultiplexed FASTQ files were generated with Casava 1.8.2.

### Example 3—RNAseq Data Acquisition, Quality Control, and Processing

RNAseq reads were aligned to the Ensembl release 76 assembly with STAR version 2.0.4b (17). Gene counts were derived from the number of uniquely aligned unambiguous reads by Subread:feature Count version 1.4.5. Transcript counts were produced by Sailfish version 0.6.3. Sequencing performance was assessed for total number of aligned reads, total number of uniquely aligned reads, genes and transcripts detected, ribosomal fraction known junction saturation and read distribution over known gene models with RSeQC version 2.3.

All gene-level and transcript counts were then imported into the R/Bioconductor package EdgeR and TMM normalization size factors were calculated to adjust for samples for differences in library size. Genes or transcripts not expressed in any sample were excluded from further analysis. The TMM size factors and the matrix of counts were then imported into R/Bioconductor package Limma and weighted likelihoods based on the observed mean-variance relationship of every gene/transcript were then calculated for all samples with the Voom function. Performance of the samples was assessed with a spearman correlation matrix and multi-dimensional scaling plots. Gene/transcript performance was assessed with plots of residual standard deviation of every gene to their average log-count with a robustly fitted trend line of the residuals. Generalized linear models with robust dispersion estimates were then created to test for gene/transcript level differential expression. Differentially expressed genes and transcripts were then filtered for FDR adjusted p-values less than or equal to 0.05.

### Example 4—Quantitative RT-PCR

Quantitative RT-PCR: Cells were cultured as above. Total RNA was isolated from 1×106 cells using RNeasy Mini Kit (Qiagen, Germantown, Md.) following the manufacturer's protocol. Single stranded cDNA was synthesized from fresh total RNA using SuperScript III First-Strand Synthesis System for RT-PCR following the manufacturer's instructions (Invitrogen, Carlsbad, Calif.). Real time quantitative PCR was performed with SsoAdvanced SYBR Green Supermix (Biorad, Hercules, Calif.) according to manufacturer's protocol. PCR primers were pre-designed and purchased from Biorad (Hercules, Calif.)

### Example 4—Statistical Tests

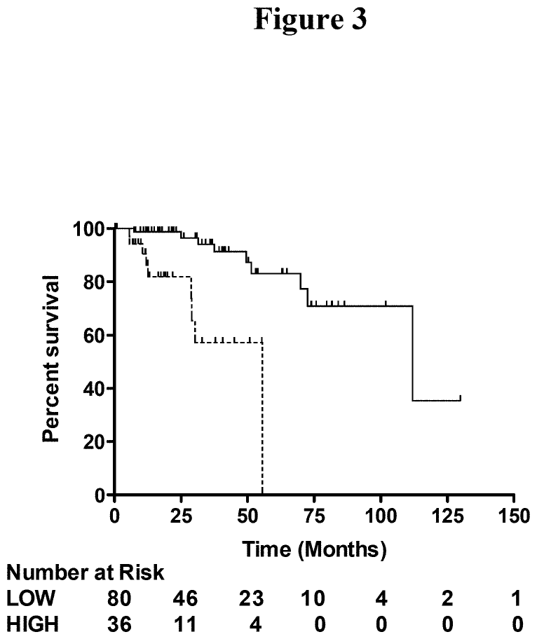

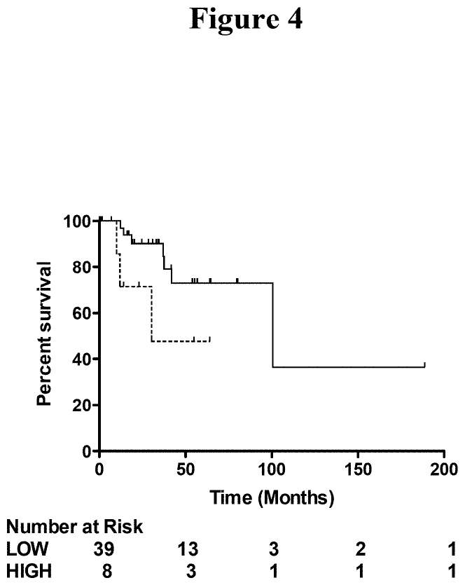

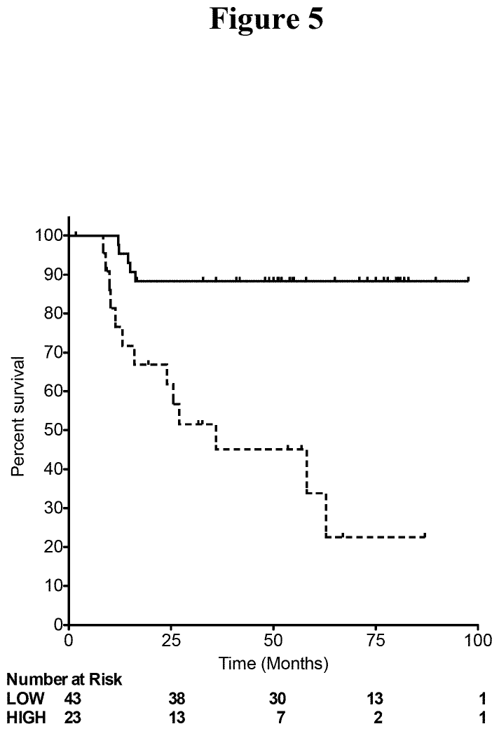

Kaplan Meier plots were generated using the software program Prism. Statistical significance was determined using both Mantel-Cox and Gehan-Breslow-Wilcoxon Tests (p<0.05). FIG. 3 is a chart showing a Kaplan Meier plot of those with ≤1 alteration (solid line) and ≥2 alterations (dashed line) in the HER2+ population (N=120) of TCGA Cell Study, p<0.05. FIG. 4 is a chart showing a Kaplan Meier plot of those with ≤1 alteration (solid line) and ≥2 alterations (dashed line) in the HER2+ population (N=49) of TCGA Nature Study, p<0.05. FIG. 5 is a chart showing a Kaplan Meier of the 6-gene signature validated in the HER2+ population (N=150) in KM Plotter, p<0.05, (solid line) is survival of patients with low expression and (dashed line) is survival of patients with high expression.

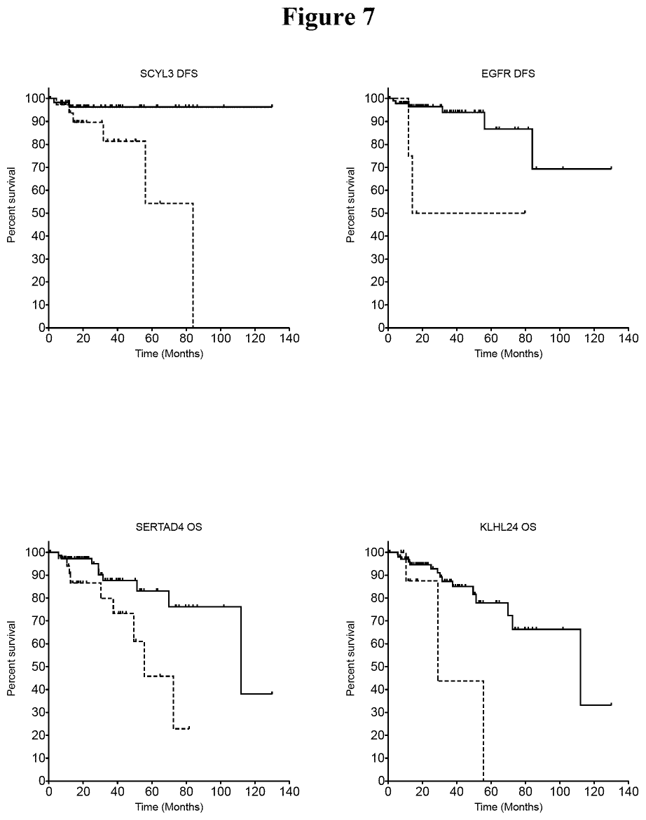

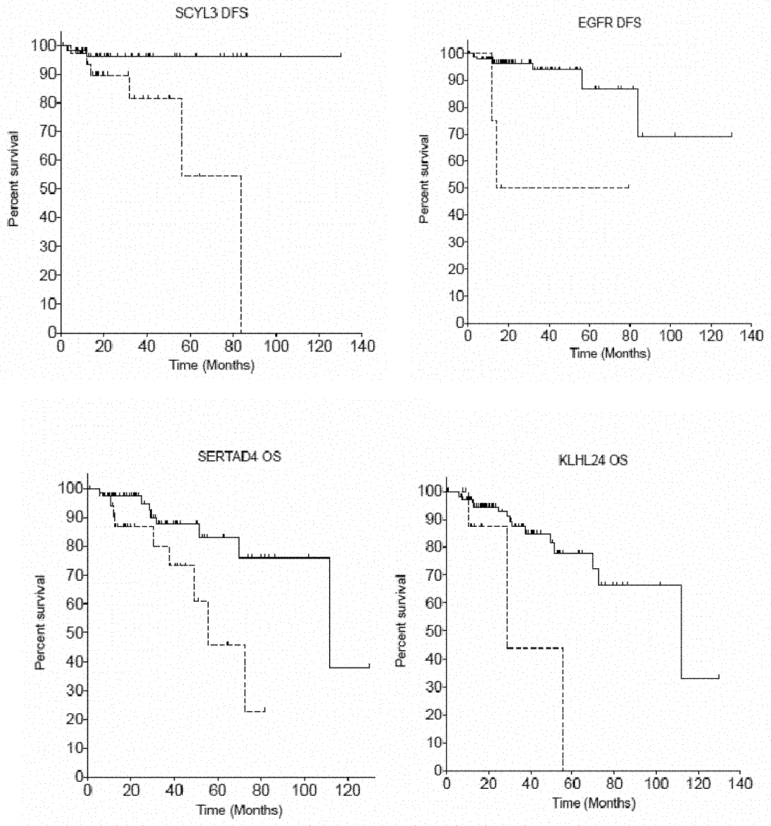

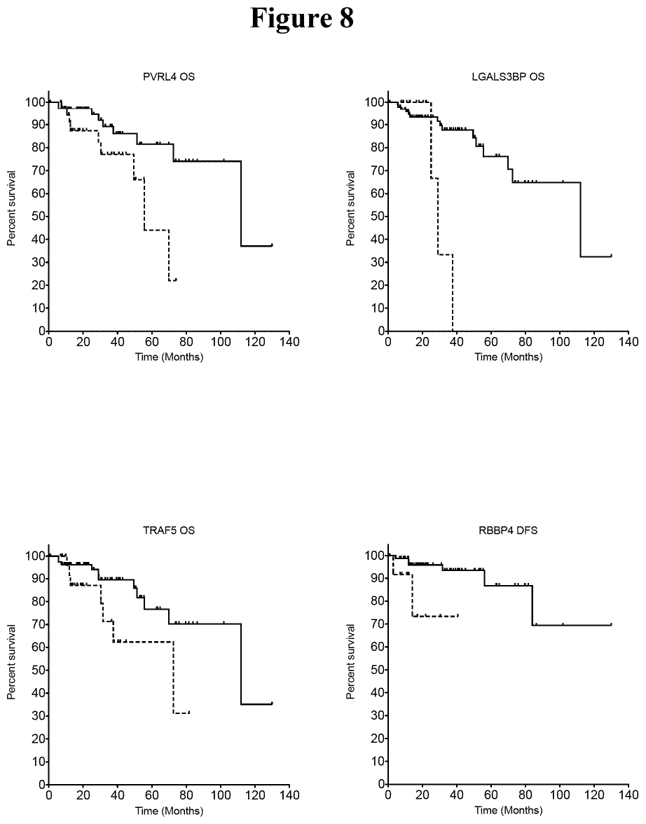

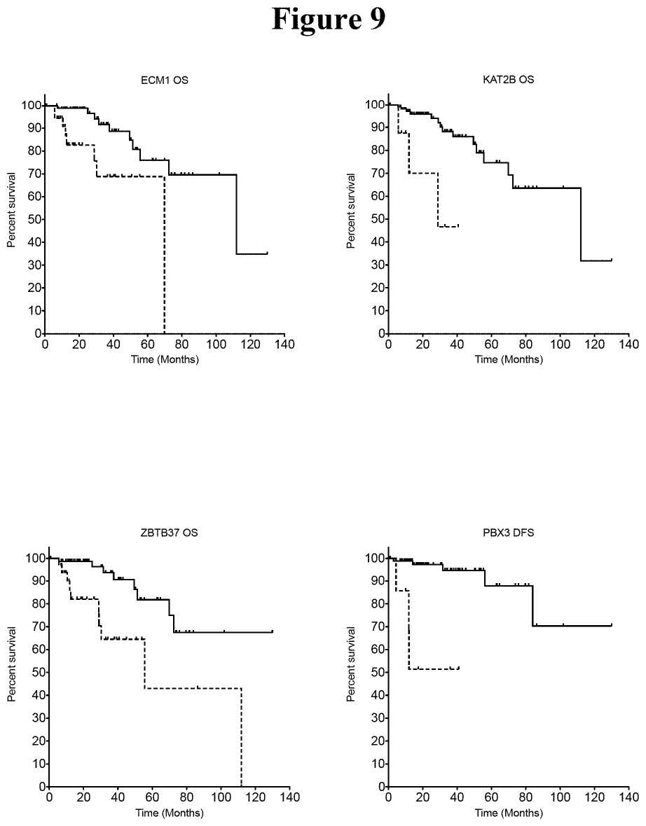

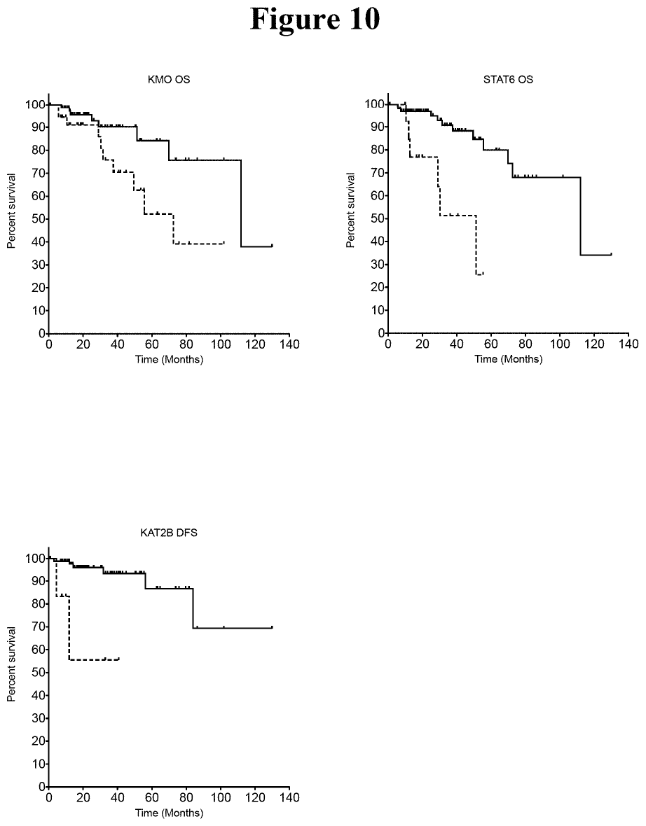

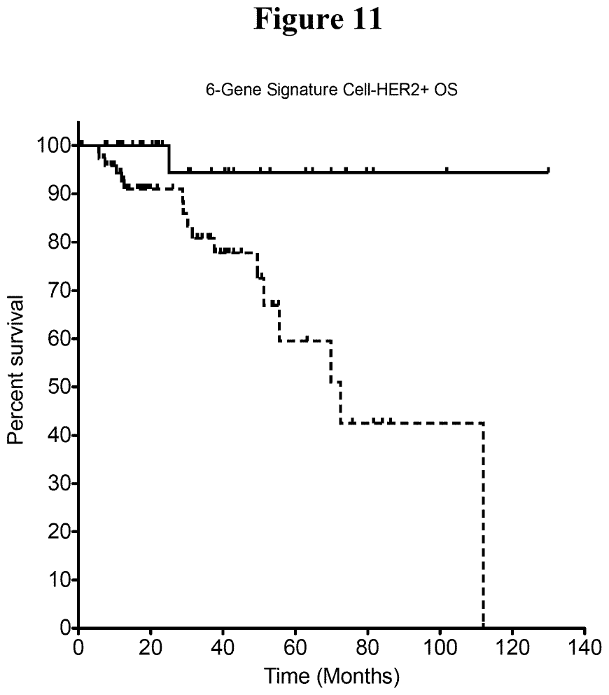

FIGS. 7 and 8 are graphs of individual Kaplan Meier plots for candidate genes. FIGS. 9 and 10 are graphs of Kaplan Meier plots for genes validated in Cell 2015 Study (27), HER2+ population N=120, p<0.05, (dashed line) is survival of patients with gene alterations and (solid line) is survival of patients without gene alterations. FIG. 11 is a Kaplan Meier plot of overall survival (OS) of 6-signature as validated in HER2+ population (N=120), p<0.05, of Cell 2015 study. (dashed line) is survival of patients with gene alterations and (solid line) is survival of patients without gene alterations.

### Example 5—Use of Next Generation Sequencing to Identify Differentially Expressed Genes (“DEGs”) Associated with Trastuzumab Resistance

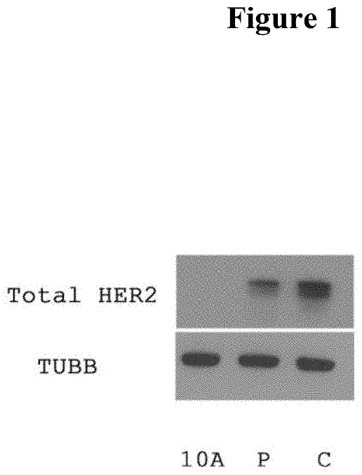

It is known that the loss of ERBB2 expression can lead to trastuzumab resistance (18). Consequently, we confirmed that the expression of the ERBB2 protein was stable in parent and resistant clones (FIG. 1).

We reasoned that the isogenic nature of the cellular clones used in this study would enrich for DEGs that were most associated with trastuzumab resistance. This approach eliminated “passenger” genetic alterations by selecting only statistically significant DEGs (p<0.05), leaving us with 3,241. We utilized two independent workflows to identify candidate genes that may play a role in trastuzumab resistance.

In the first workflow, we filtered the DEGs to only include those satisfying the criteria −0.5>Log FC>0.5. Next, we used the online tool cBioportal to identify DEGs that occur in at least 25% of HER2+ breast cancer patients, using The Cancer Genome Atlas (TCGA), which left us with 107 DEGs (19-21). TCGA study molecularly characterized breast invasive lobular and invasive ductal carcinomas, which included all subtypes of breast cancer.

In the second workflow, we used Ingenuity Pathway Analysis (IPA; Qiagen, Germantown, Md.) to filter all DEGs (3,241) through enrichment analysis to identify networks of interest. Of these pathways, eukaryotic initiation factor (EIF) signaling (p=1.7×10−13) was noteworthy because the significance of this pathway overshadowed the other pathways by nearly 10 orders of magnitude. EIF signaling was expanded to include networks featuring EIF2, EIF3, ribosomal 40s, ribosomal 60s and ribonucleotide reductase (RnR). RnR was subsequently expanded further to incorporate RNA Polymerase II. IPA also identified several top upstream regulators, lysine specific demethylase 5B (KDM5B) and estrogen receptor (ESR1). Genes incorporated in these networks were retained if the Log fold change (FC) observed between the trastuzumab sensitive and resistant clones was −0.9>Log FC>0.9, leaving 117 DEGs.

The resulting list of combined genes from both workflows was further filtered based on agreement with publically-available clinical data. We added the condition that the observed trend in RNA expression from RNAseq of a given DEG had to agree with the trend observed in publically-available datasets for RNAseq and/or copy number of breast cancer patient specimens. The remaining candidate genes were further scrutinized by both the Log-rank Mantel-Cox (FIGS. 7 and 8) and Grehan-Breslow-Wilcoxon tests (p<0.05) in OS and/or DFS (19, 20).

### Example 6—Validation of Candidate Genes Using Independent Samples

The candidate genes in the study were identified from an isogenic cell line model using RNAseq to measure changes in gene expression of trastuzumab-resistant vs. -sensitive clones. In order to determine the clinical relevance of our candidate genes, we turned to TCGA. We identified six genes whose individual and collective changes in either expression and/or copy number were predictive of DFS or OS in HER2+ breast cancer patients (21).

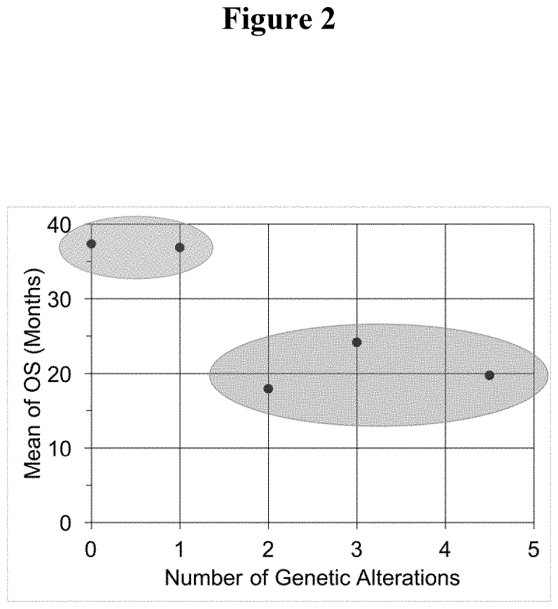

Next, we looked to further validate our gene signature using a second, independent cohort of patient samples to determine if the statistically significant changes in OS were genuine. Therefore, we turned to TCGA study published in Nature 2012. TCGA project is made up of two studies with independent patient samples published in 2012 and 2015. We stratified HER2+ patients from TCGA 2012 study based on those with ≤1 and ≥2 alterations from our six gene signature and plotted OS using Kaplan Meier analysis. Interestingly, we observed a statistically significant difference in OS (p=0.0374) using this independent dataset (FIG. 4). We report a median survival of 100 and 30 months for patients with ≤1 and ≥2 genetic alterations from our signature, respectively. In order to confirm that this predictive power is unique to HER2+ patients, we applied it to HER2− patients and no statistically-significant difference in OS was observed. FIG. 2 is a plot showing the number of genetic alterations from the six gene signature plotted against mean overall survival (months).

In order to further validate our findings, we turned to the kmplot.com to stratify the HER2+ patients (N=150) carrying at least one DEG from the six gene candidate list into two groups, those with high expression and those with low expression (22). Here, we observed statistically-significant changes in both DES and distant metastasis free survival (DMFS) (FIG. 5). It is important to note that when the 6 gene signature was applied to the HER2-negative patient populations of both the KM Plotter data and TCGA studies. No statistically significant difference was observed in either OS or DFS using both the Mantel-Cox and Wilcoxon statistical tests for the TCGA studies. However, even though there was no statistical significance for DMFS, there were statistically significant differences for DFS and OS in the KM Plotter data.

### Example 7—Validation of Candidate Genes by qPCR

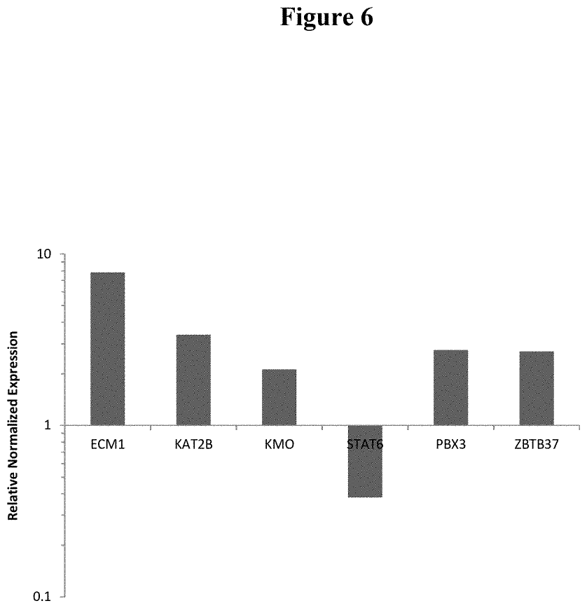

To confirm the gene expression changes observed by RNAseq, we turned to qPCR. Differential expression of all six genes in our signature was validated by qPCR (FIG. 6).

### Example 8—Identification of Gene Expression Signature

Trastuzumab revolutionized the treatment of HER2+ breast cancers, but most patients experience either de novo or acquired resistance (23). Mechanisms of trastuzumab resistance have been studied exhaustively in pre-clinical and clinical studies with disappointing results (11-13). Therefore, there is a need to understand the underlying mechanisms contributing to resistance.

The inventors reasoned that one explanation for this disconnect may be the screening strategies for resistance mechanisms. Human tumors exhibit inter-tumor heterogeneity and similarly, cell culture models also exhibit heterogeneity. The inventors utilized an isogenic cell line model to control and limit the extent of heterogeneity. This methodology helps to identify and enrich for genetic “drivers” of trastuzumab resistance, while limiting the number of “passenger” alterations.

The use of RNAseq can produce an overwhelming number of target genes, which obfuscates the interpretation of the data. The isogenic nature of the model system greatly reduced the number of “passenger” DEGs and enriched for “drivers” of trastuzumab resistance. However, 3,241 DEGs remained. Thus, filtering strategies were required to enrich for the most favorable genes. Two different workflows allowed identification of relevant DEGs.

At the same time, one does not want to lose clinically interesting genes. Since data analyzed by multiple bioinformatic pipelines are often not in agreement with each other, we eliminated genes that were not differentially expressed in both platforms (24). This reduced the likelihood of losing relevant DEGs. This effort, coupled with qPCR validation and enrichment for clinically-significant genes greatly enriched for the most clinically interesting genes.

Drug resistance often occurs through genetic alterations in tumors that endow cells with aberrant expression of native or mutant versions of genes. There are likely several genetic alterations that can cause drug resistance. We were interested in identifying biomarkers that could address the problem of overtreatment in HER2+ breast cancer patients. The National Comprehensive Cancer Network recommends that HER2+ breast cancer patients receive frontline treatment with trastuzumab and pertuzumab, supplemented with a taxane or taxotere.

However, approximately 20% of HER2+ breast cancer patients experience a potent and durable response to single-agent trastuzumab, meaning that some patients will respond without the need for DNA-damaging chemotherapy (5). We looked towards a genetic signature that could help identify these patients. Precedent for the success of gene signature risk assessment tools has been seen in ER+ breast cancers with the 21-gene signature, which can help select patients who require chemotherapy (25). The goal of the current study was to identify a similar set of genes, whose expression would be negative predictors of response to trastuzumab.

Using an isogenic model of trastuzumab resistance in HER2+ breast cancer cells in combination with a systems biology approach, we were able to identify a six gene signature (ECM1, KAT2B, KMO, PBX3, STAT6 and ZBTB37) that predicts for poor overall survival and disease-free survival.

Kynurenine 3-monooxygenase (KMO) was found to be one of the genes with predictive power. It is a NADPH dependent flavin monooxygenase that catalyzes the hydroxylation of L-kynurenine to produce L-3-hydroxykynurenine. Overexpression of KMO has been shown to promote proliferation, migration and invasion in human hepatocellular carcinoma cells (26). Our study too saw an increase in expression of KMO through RNAseq and qPCR further suggesting its influence in breast cancer. Its pathway promotes the activation of cytokine-mediated changes in the presence of inflammatory stimuli such as infection (27). A major mechanism of action for trastuzumab is through antibody dependent cell mediated toxicity (ADCC), which acts like an inflammatory response. It is possible that elevated expression of KMO counteracts ADCC by promoting cytokine-mediated growth and contributing to trastuzumab resistance.

We found ECM1 to be one of the most highly expressed genes in our RNAseq and qPCR data. ECM1 is a glycoprotein overexpressed in many carcinomas and has been shown to play a possible role in breast carcinogenesis (28). It is specifically implicated in promoting cell proliferation, angiogenesis and differentiation in invasive ductal breast cancer (29, 30). A study by Wang et al. found that the expression of ECM1 was correlated with tumor metastases, with 76% of ECM1-expressing primary breast samples developing metastases versus non-ECM1-expressing tumors, where only 33% of tumors developed metastases (30). ECM1 has also been implicated in the Warburg Effect, where increased expression of ECM1 in a trastuzumab resistant clone of BT474 cells, sensitized cells to the uptake of glucose (28).

Zinc finger and BTB domain containing 37 (ZBTB37) was found to be upregulated by RNAseq in our trastuzumab resistant clone when compared to the trastuzumab sensitive clone. Using a yeast two-hybrid system or tandem affinity purification followed by mass spectrometry, ZBTB37 has been found to interact with the heat shock protein HSPB1, the transcription factor FOXP3 and SH3BP4 (31-33). In cancer, it ZBTB37 is thought to be a tumor suppressor gene, as it is a negative regulator of the GTPase-mTORC1 signaling (34).

The remaining genes all have a role in gene transcription and include: K(lysine) acetyltransferase 2B (KAT2B), Pre-leukemia transcription factor 3 (PBX3), and Signal transducer and activator of transcription 6 (STATE). Several studies have linked dysregulation of transcriptions factors to drug resistance in tumors and cell lines (35-37). KAT2B is a histone acetyltransferase and transcriptional coactivator associated with TP53 (38, 39). Overexpression of KAT2B has been reported in alveolar rhabdomyosarcoma and pancreatic cell lines (40, 41). A study by He et al. found that silenced KAT2B in DU145 cells inhibited cell viability, thus normal or increased expression may promote proliferation (42). We observed upregulation of KAT2B in the trastuzumab resistant clone, which is in agreement with the aforementioned studies. Taken together, KAT2B is associated with a transformative cancer phenotype that promotes aberrant cellular growth.

PBX3 is a member of the PBX family of transcription factors known to increase DNA-binding and transcriptional activity of homeobox (HOX) proteins. HOXB7 has been known to confer tamoxifen resistance through increased signaling of the EGFR pathway and elevated levels are associated with a poorer outcome in patients diagnosed with HER2+ breast cancer (43, 44). Aberrant EGFR signaling has been implicated in trastuzumab resistance, which may explain an indirect role of PBX3 in trastuzumab resistance. Overexpression of PBX3 and other HOX genes have been reported in malignant prostate tissue and in poor survival outcomes in AML patients (45, 46). Taken together with our current data, increased expression of PBX3 leads to unabated growth and decreased survival in cancer patients.

The last transcription factor from our gene signature is STAT6, which is a member of the STAT family. In a recent preclinical study, Zhu et al. reported that STAT6 is a negative regulator of cell proliferation by induction of p21 and p27 expression, which are known to play a role in tumor suppression (47). Interestingly, RNAseq and qPCR data from the current study found that STAT6 was down regulated in the trastuzumab resistant clones, which is in line with the above study. It is possible that downregulation of STAT6 in the resistant clone prevents expression of p21 and p27, which leads to aberrant cell growth.

Since it is possible that our biomarker panel may predict for poor outcome of single agent trastuzumab treatment in HER2+ patients, then it would be beneficial to find drug alternatives and/or trastuzumab combination therapy that could be tailored to this subpopulation. Another HER2-targeted agent is lapatinib, which is a small molecule tyrosine kinase inhibitor. We determined the gene expression values of our six-gene signature in publically available RNAseq and gene expression microarray datasets of isogenic clones of BT474 (GSE16179) and SKBR3 (GSE81546) conditioned to exhibit resistance to lapatinib (48, 49). In the lapatinib resistant BT474 cells, gene expression of ECM1, PBX3, and ZBTB37 were significantly altered compared to parental clones. In the SKBR3 cellular clones, only four of our six-genes were measured. Of these four genes, a statistically significant change in KAT2B and PBX3 expression were observed between parent and resistant clones. Our predictive signature required that only one gene be altered to predict for poorer outcome. Thus, this gene signature was predictive in two independent lapatinib-resistant datasets using different platforms for gene expression determination.

A study by Kim et al. used a bioinformatics approach to identify 33 genes, whose altered expression was associated with trastuzumab resistance (50). They utilized a publically available microarray dataset for the same cell lines used in this study (GSE15043). However, none of our six genes appeared in Kim et al.'s candidate list. This could be explained by the differences in the platforms and data analyses used across the studies. Their study utilized microarray analysis, while we employed RNAseq. The pipelines used to analyze microarray vs. RNAseq data can result in poor overlap across data sets (24).

To conclude, we describe herein a six-gene signature that predicts poor overall and disease-free survival in HER2+ breast cancer patients. We were able to validate these predictions in four publically available datasets of human tumors and preclinical models of drug resistance. There is also strong evidence indicating that patients who carry this gene signature will not respond to less aggressive treatments using single agent trastuzumab and endocrine therapies. Embodiments of the invention will be further described in the following examples, which do not limit the scope of the invention described in the claims.

### REFERENCES

1. Slamon D J, Clark G M, Wong S G, Levin W J, Ullrich A, McGuire W L. Human breast cancer: correlation of relapse and survival with amplification of the HER-2/neu oncogene. Science. 1987; 235:177-82.
2. Slamon D J, Godolphin W, Jones L A, Holt J A, Wong S G, Keith D E, et al. Studies of the HER-2/neu proto-oncogene in human breast and ovarian cancer. Science. 1989; 244:707-12.
3. Carter P, Presta L, Gorman C M, Ridgway J B, Henner D, Wong W L, et al. Humanization of an anti-p185HER2 antibody for human cancer therapy. Proc Natl Acad Sci USA. 1992; 89:4285-9.
4. Vogel C L, Cobleigh M A, Tripathy D, Gutheil J C, Harris L N, Fehrenbacher L, et al. Efficacy and safety of trastuzumab as a single agent in first-line treatment of HER2-overexpressing metastatic breast cancer. Journal of clinical oncology: official journal of the American Society of Clinical Oncology. 2002; 20:719-26.
5. Cobleigh M A, Vogel C L, Tripathy D, Robert N J, Scholl S, Fehrenbacher L, et al. Multinational study of the efficacy and safety of humanized anti-HER2 monoclonal antibody in women who have HER2-overexpressing metastatic breast cancer that has progressed after chemotherapy for metastatic disease. J Clin Oncol. 1999; 17:2639-48.
6. Slamon D J, Leyland-Jones B, Shak S, Fuchs H, Paton V, Bajamonde A, et al. Use of chemotherapy plus a monoclonal antibody against HER2 for metastatic breast cancer that overexpresses HER2. N Engl J Med. 2001; 344:783-92.
7. Piccart-Gebhart M J, Procter M, Leyland-Jones B, Goldhirsch A, Untch M, Smith I, et al. Trastuzumab after adjuvant chemotherapy in HER2-positive breast cancer. N Engl J Med. 2005; 353:1659-72.
8. Romond E H, Perez E A, Bryant J, Suman V J, Geyer C E, Jr., Davidson N E, et al. Trastuzumab plus adjuvant chemotherapy for operable HER2-positive breast cancer. N Engl J Med. 2005; 353:1673-84.
9. Joensuu H, Kellokumpu-Lehtinen P L, Bono P, Alanko T, Kataja V, Asola R, et al. Adjuvant docetaxel or vinorelbine with or without trastuzumab for breast cancer. N Engl J Med. 2006; 354:809-20.
10. Carlson R W, Allred D C, Anderson B O, Burstein H J, Carter W B, Edge S B, et al. Breast cancer. Clinical practice guidelines in oncology. J Natl Compr Canc Netw. 2009; 7:122-92.

11. Rexer B N, Arteaga C L. Intrinsic and acquired resistance to HER2-targeted therapies in HER2 gene-amplified breast cancer: mechanisms and clinical implications. Crit Rev Oncog. 2012; 17:1-16.
12. Perez E A, Dueck A C, McCullough A E, Chen B, Geiger X J, Jenkins R B, et al. Impact of PTEN protein expression on benefit from adjuvant trastuzumab in early-stage human epidermal growth factor receptor 2-positive breast cancer in the North Central Cancer Treatment Group N9831 trial. J Clin Oncol. 2013; 31:2115-22.
13. Nagata Y, Lan K H, Zhou X, Tan M, Esteva F J, Sahin A A, et al. PTEN activation contributes to tumor inhibition by trastuzumab, and loss of PTEN predicts trastuzumab resistance in patients. Cancer Cell. 2004; 6:117-27.
14. Chan C T, Metz M Z, Kane S E. Differential sensitivities of trastuzumab (Herceptin)-resistant human breast cancer cells to phosphoinositide-3 kinase (PI-3K) and epidermal growth factor receptor (EGFR) kinase inhibitors. Breast cancer research and treatment. 2005; 91:187-201.
15. Torrance C J, Agrawal V, Vogelstein B, Kinzler K W. Use of isogenic human cancer cells for high-throughput screening and drug discovery. Nat Biotechnol. 2001; 19:940-5.
16. Abukhdeir A M, Vitolo M I, Argani P, De Marzo A M, Karakas B, Konishi H, et al. Tamoxifen-stimulated growth of breast cancer due to p21 loss. Proceedings of the National Academy of Sciences. 2008; 105:288-93.
17. Dobin A, Davis C A, Schlesinger F, Drenkow J, Zaleski C, Jha 5, et al. STAR: ultrafast universal RNA-seq aligner. Bioinformatics. 2013; 29:15-21.
18. Elster N, Collins D M, Toomey S. Crown J, Eustace A J, Hennessy B T. HER2-family signalling mechanisms, clinical implications and targeting in breast cancer. Breast cancer research and treatment. 2015; 149:5-15.
19. Gao J, Aksoy B A, Dogrusoz U, Dresdner G, Gross B, Sumer S O, et al. Integrative analysis of complex cancer genomics and clinical profiles using the cBioPortal. Sci Signal. 2013; 6:p11.
20. Cerami E, Gao J, Dogrusoz U, Gross B E, Sumer S O, Aksoy B A, et al. The cBio cancer genomics portal: an open platform for exploring multidimensional cancer genomics data. Cancer Discov. 2012; 2:401-4.
21. Ciriello G, Gatza M L, Beck A H, Wilkerson M D, Rhie S K, Pastore A, et al. Comprehensive Molecular Portraits of Invasive Lobular Breast Cancer. Cell. 2015; 163:506-19.
22. Gyorffy B, Lanczky A, Eklund A C, Denkert C, Budczies J, Li 0, et al. An online survival analysis tool to rapidly assess the effect of 22,277 genes on breast cancer prognosis using microarray data of 1,809 patients. Breast cancer research and treatment. 2010; 123:725-31.
23. Ahmad S, Gupta S, Kumar R, Varshney G C, Raghava G P. Herceptin resistance database for understanding mechanism of resistance in breast cancer patients. Sci Rep. 2014; 4:4483.
24. O'Rawe J, Jiang T, Sun G, Wu Y, Wang W, Hu J, et al. Low concordance of multiple variant-calling pipelines: practical implications for exome and genome sequencing. Genome Med. 2013; 5:28.
25. Sparano J A, Gray R J, Makower D F, Pritchard K I, Albain K S, Hayes D F, et al. Prospective Validation of a 21-Gene Expression Assay in Breast Cancer. New England Journal of Medicine. 2015; 373:2005-14.
26. Jin H, Zhang Y, You H, Tao X, Wang C, Jin G, et al. Prognostic significance of kynurenine 3-monooxygenase and effects on proliferation, migration, and invasion of human hepatocellular carcinoma. Sci Rep. 2015; 5:10466.
27. Dantzer R, O'Connor J C, Lawson M A, Kelley K W. Inflammation-associated depression: from serotonin to kynurenine. Psychoneuroendocrinology. 2011; 36:426-36.
28. Lee K M, Nam K, Oh S, Lim J, Lee T, Shin I. ECM1 promotes the Warburg effect through EGF-mediated activation of PKM2. Cell Signal. 2015; 27:228-35.
29. Han Z, Ni J, Smits P, Underhill C B, Xie B, Chen Y, et al. Extracellular matrix protein 1 (ECM1) has angiogenic properties and is expressed by breast tumor cells. FASEB J. 2001; 15:988-94.
30. Wang L, Yu J, Ni J, Xu X M, Wang J, Ning H, et al. Extracellular matrix protein 1 (ECM1) is over-expressed in malignant epithelial tumors. Cancer letters. 2003; 200:57-67.
31. Katsagiannou M, Andrieu C, Baylot V, Baudot A, Dusetti N J, Gayet O, et al. The functional landscape of Hsp27 reveals new cellular processes such as DNA repair and alternative splicing and proposes novel anticancer targets. Mol Cell Proteomics. 2014; 13:3585-601.
32. Li X, Wang W, Wang J, Malovannaya A, Xi Y, Li W, et al. Proteomic analyses reveal distinct chromatin-associated and soluble transcription factor complexes. Mol Syst Biol. 2015; 11:775.
33. Thalappilly S, Suliman M, Gayet O, Soubeyran P, Hermant A, Lecine P, et al. Identification of multi-SH3 domain-containing protein interactome in pancreatic cancer: a yeast two-hybrid approach. Proteomics. 2008; 8:3071-81.
34. Kim Y-M, Stone M, Hwang T H, Kim Y-G, Dunlevy J R, Griffin T J, et al. SH3BP4 is a negative regulator of amino acid-Rag GTPase-mTORC1 signaling. Mol Cell. 2012; 46:833-46.
35. Kohno K, Uchiumi T, Niina I, Wakasugi T, Igarashi T, Momii Y, et al. Transcription factors and drug resistance. Eur J Cancer. 2005; 41:2577-86.
36. Dittmer J. The role of the transcription factor Ets1 in carcinoma. Semin Cancer Biol. 2015; 35:20-38.
37. Calaf G M, Zepeda A B, Castillo R L, Figueroa C A, Arias C, Figueroa E, et al. Molecular aspects of breast cancer resistance to drugs (Review). Int J Oncol. 2015; 47:437-45.
38. Jin Q, Zhuang L, Lai B, Wang C, Li W, Dolan B, et al. Gcn5 and PCAF negatively regulate interferon-beta production through HAT-independent inhibition of TBK1. EMBO Rep. 2014; 15:1192-201.
39. Liu L, Scolnick D M, Trievel R C, Zhang H B, Marmorstein R, Halazonetis T D, et al. p53 sites acetylated in vitro by PCAF and p300 are acetylated in vivo in response to DNA damage. Mol Cell Biol. 1999; 19:1202-9.
40. Bharathy N, Suriyamurthy S, Rao V K, Ow J R, Lim H J, Chakraborty P, et al. P/CAF mediates PAX3-FOXO1 dependent oncogenesis in alveolar rhabdomyosarcoma. J Pathol. 2016.
41. Ansari D, Andersson R, Bauden M P, Andersson B, Connolly J B, Welinder C, et al. Protein deep sequencing applied to biobank samples from patients with pancreatic cancer. J Cancer Res Clin Oncol. 2015; 141:369-80.
42. He W, Zhang M G, Wang X J, Zhong S, Shao Y, Zhu Y, et al. KAT5 and KAT6B are in positive regulation on cell proliferation of prostate cancer through PI3K-AKT signaling. Int J Clin Exp Pathol. 2013; 6:2864-71.
43. Morgan R, Boxall A, Harrington K J, Simpson G R, Gillett C, Michael A, et al. Targeting the HOX/PBX dimer in breast cancer. Breast cancer research and treatment. 2012; 136:389-98.
44. Chen H, Lee J S. Liang X, Zhang H, Zhu T, Zhang Z, et al. Hoxb7 inhibits transgenic HER-2/neu-induced mouse mammary tumor onset but promotes progression and lung metastasis. Cancer Res. 2008; 68:3637-44.
45. Ramberg H, Alshbib A, Berge V, Svindland A, Tasken K A. Regulation of PBX3 expression by androgen and Let-7d in prostate cancer. Mol Cancer. 2011; 10:50.
46. Bullinger L, Dohner K, Bair E, Frohling S, Schlenk R F, Tibshirani R, et al. Use of gene-expression profiling to identify prognostic subclasses in adult acute myeloid leukemia. N Engl J Med. 2004; 350:1605-16.
47. Wei M, Liu B, Gu Q, Su L, Yu Y, Zhu Z. Stat6 cooperates with Sp1 in controlling breast cancer cell proliferation by modulating the expression of p21(Cip1/WAF1) and p27 (Kip1). Cell Oncol (Dordr). 2013; 36:79-93.
48. Deblois G, Smith H W, Tam I S, Gravel S P, Caron M, Savage P, et al. ERRalpha mediates metabolic adaptations driving lapatinib resistance in breast cancer. Nat Commun. 2016; 7:12156.
49. Liu L, Greger J, Shi H, Liu Y, Greshock J, Annan R, et al. Novel mechanism of lapatinib resistance in HER2-positive breast tumor cells: activation of AXL. Cancer Res. 2009; 69:6871-8.
50. Nam S, Chang H R, Jung H R, Gim Y, Kim N Y, Grailhe R, et al. A pathway-based approach for identifying biomarkers of tumor progression to trastuzumab-resistant breast cancer. Cancer letters. 2015; 356:880-90.

We claim:

1. A method for treating a cancer in a subject, the method comprising:
determining a gene expression signature based on expression level, copy number or promotor methylation of at least two genes selected from the group consisting of ECM1, KAT2B, KMO, PBX3, STAT6 and ZBTB37, and
administering a treatment strategy to the subject based on the gene expression signature.

2. The method of claim 1, wherein the gene expression signature is based on expression level, copy number or promotor methylation of at least three genes selected from the group consisting of ECM1, KAT2B, KMO, PBX3, STAT6 and ZBTB37.

3. The method of claim 2, wherein the gene expression signature is based on expression level, copy number or promotor methylation of at least four genes selected from the group consisting of ECM1, KAT2B, KMO, PBX3, STAT6 and ZBTB37.

4. The method of claim 3, wherein the gene expression signature is based on expression level, copy number or promotor methylation of genes ECM1, KAT2B, KMO, PBX3, STAT6 and ZBTB37.

5. The method of claim 1, wherein the treatment strategy comprises administering a less harsh treatment strategy when the gene expression signature is indicative of at least two of: normal or decreased expression levels of ECM1, KAT2B, KMO, PBX3 and ZBTB37 and normal or increased expression level of STAT6.

6. The method of claim 5, wherein the less harsh treatment strategy comprises the administration of single-agent trastuzumab therapy.

7. The method of claim 5 comprising administering the treatment strategy comprising single-agent trastuzumab therapy when the gene expression signature is indicative of at least four of: normal or decreased expression levels of ECM1, KAT2B, KMO, PBX3 and ZBTB37 and normal or increased expression level of STAT6.

8. The method of claim 7 comprising administering the treatment strategy comprising single-agent trastuzumab therapy when the gene expression signature is indicative of: normal or decreased expression levels of ECM1, KAT2B, KMO, PBX3 and ZBTB37 and normal or increased expression level of STAT6.

9. The method of claim 1 comprising administering the treatment strategy comprising a harsh treatment strategy when the gene expression signature is indicative of at least two of: elevated expression levels of ECM1, KAT2B, KMO, PBX3 and ZBTB37 and decreased expression level of STAT6.

10. The method of claim 9 comprising administering the treatment strategy comprising a harsh treatment strategy when the gene expression signature is indicative of at least three of: elevated expression levels of ECM1, KAT2B, KMO, PBX3 and ZBTB37 and decreased expression level of STAT6.

11. The method of claim 10 comprising administering the treatment strategy comprising a harsh treatment strategy when the gene expression signature is indicative of at least five of: elevated expression levels of ECM1, KAT2B, KMO, PBX3 and ZBTB37 and decreased expression level of STAT6.

12. The method of claim 11 comprising administering the treatment strategy comprising a harsh treatment strategy when the gene expression signature is indicative of elevated expression levels of ECM1, KAT2B, KMO, PBX3 and ZBTB37 and decreased expression level of STAT6.

13. The method of claim 9, wherein the harsh treatment strategy comprises chemotherapy using one or more drugs selected from the group consisting of doxorubicin, cyclophosphamide, doxil, eribulin mesylate, ixabepilone, vinorelbine, gemcitabine, cis-platinum, carboplatin, nab-paclitaxel, paclitaxel, docetaxel, capecitabine, metronomic methotrexate, cyclophosphamide, oral VP-16 and bevacizumab.

14. The method of claim 1, wherein the cancer is selected from the group consisting of breast cancer, HER2+ breast cancer, bladder cancer, carcinoid cancer, colon cancer, ductal carcinoma in situ (DCIS), endometrium cancer, head/neck cancer, kidney cancer, lung cancer, melanoma, ovarian cancer, phylloides cancer, prostate cancer, stomach cancer, testicular cancer and thyroid cancer.

15. The method of claim 14, there the cancer is breast cancer.

16. The method of claim 15, wherein the breast cancer is HER2+ breast cancer.
